(19) United States
(12) Patent Application Publication
(10) Pub. No.: US 2006/0264913 A1
(43) Pub. Date: Nov. 23, 2006

(54) Implantable flow regulator with failsafe mode and reserve drug supply

(76) Inventors: Andrew I. Poutiatine, Menlo Park, CA (US); Edward M. Gillis, Cupertino, CA (US); Pierre-Alain Mausli, Cheseaux (CH); Volker Gass, Penthalaz (CH)

Correspondence Address: DURECT CORPORATION, 2 Results Way, Cupertino, CA, 95014, US

(21) Appl. No.: 11/476,372

(22) Filed: Jun. 27, 2006

Related U.S. Application Data
(63) Continuation of application No. 10/525,882, filed on Feb. 24, 2005.

(51) Int. Cl. A61K 9/22

(52) U.S. Cl. 604/891.1; 604/250

(57) ABSTRACT

An implantable flow regulator (22) for regulating flow of a drug along a drug delivery pathway (14) from a drug source to a treatment site within a subject includes a movable diversion member (24) and an actuator. The movable diversion member (24) is operably coupled to a delivery pathway (14). The actuator is actuatable to move the diversion member (24) between at least a first mode position where the diversion member (24) restricts flow through the delivery pathway (14) (i.e., delivery pathway is “closed”), and a second mode position where the diversion member (24) is position to allow flow through the delivery pathway (14) (i.e., delivery pathway is “open”). Supplying power to actuate the actuator moves the diversion member (24) from its normally biased first mode or “closed” position, which is also a failsafe position, toward its second mode or “open” position. In the “open” position, drug is supplied to target sites, typically from a drug supply reserve within the regulator.

### FIELD

This application relates to implantable drug delivery devices and methods, and in particular, to flow regulators that are selectively changeable from a failsafe diversion mode to a delivery mode during which drug is delivered from a reserve in the regulator on demand to a treatment site or sites.

### BACKGROUND

Many diseases or indications require long term, chronic delivery of drugs or agents to a patient, e.g., cancer, arthritis, heart disease, diabetes, etc. Long term delivery of drugs or agents can be accomplished using drug delivery systems with components positioned external to the subject's body or, as is of interest here, components implanted within the body. These systems may include catheters, conduits or other structures to establish a delivery pathway via which drug from a source or delivery device is supplied, usually as a flowable fluid, to the treatment sites. Control of delivery according to a predetermined treatment plan usually is executed with signals from a microprocessor-based circuit connected or coupled to the system.

Some drug delivery devices can deliver the drug at a selectively variable rate. Such devices, however, tend to include complex mechanical and/or electrical components that make these devices bulky and prone to failure.

Other drug delivery devices, referred to as constant rate devices, provide for delivery of the drug at a substantially constant rate. Constant rate devices supply the drug at a pre-selected, substantially non-fluctuating rate, so the amount of drug delivered to a site is readily determinable. These constant rate devices, however, do not provide for readily changing the pre-selected delivery rate.

A change in delivery rate may be necessary for several reasons. First, the proper drug dosage may not be known prior to treatment (e.g., dose titration may be required to determine an appropriate dosage). Second, the subject may require increasing dosages (e.g., due to increasing tolerance to the drug) or decreasing dosages (e.g., as the subject's condition improves). Third, the most appropriate treatment plan may require different doses over different time periods.

A hybrid constant rate device pioneered by the present assignee (described in U.S. patent application Ser. No. 09/416,379, filed on Oct. 12, 1999 and entitled “Regulation of Drug Delivery Through Flow Diversion,” attorney docket no. DURE-O09) shows a constant rate drug source connected to a downstream implantable flow regulating device or flow regulator. The flow regulator is electrically actuatable to direct the flow that has been received from the source along one of two fluid pathways: (1) a delivery pathway to the treatment site or sites or (2) a diversion pathway to a reservoir or systemic absorption (i.e., into circulation or elsewhere). According to most treatment plans, flow is predominately directed along the diversion pathway, whereas the delivery pathway is used only a fraction of the time, since the typical doses directed to treatment site(s) are relatively small amounts, i.e., usually on the order of about 1-10 μL per day, whereas the constant rate source provides on the order of about 10 μL per day to the flow regulator (the excess 0-9 μL being diverted).

In the hybrid constant rate system, achieving delivery of drug at a desired rate quickly, and maintaining that rate substantially constant over a given treatment interval is difficult. Although the regulator may be changed from operating in the diversion mode to operating in the delivery mode relatively quickly, the response time of actual drug flow in the delivery mode is delayed. Because drug delivery during a delivery interval does not occur at a constant rate, the amount of drug delivered in an interval is difficult to quantify. If a given interval is relatively short, the amount of drug that would be delivered in that interval is even more uncertain. As a result, it can be difficult to calibrate delivery of drug as precisely as desired.

### SUMMARY

Described herein are devices and methods for regulating flow in an implantable drug delivery system that offer advantages over conventional devices and methods. The devices have a space, e.g., a chamber, within which a reserve of drug is accumulated, such that upon switching from diversion mode to delivery mode, drug is supplied from the reserve on demand with reduced delay. The resulting drug delivery rate over the delivery interval is more continuous, which allows the amount of drug delivered during delivery mode to be determined with greater precision. Following a treatment interval, the system returns to the diversion mode and the reserve is refilled to a predetermined level with drug flowing from the source within a short period. While in delivery mode and after the reserve is refilled, additional drug that flows from the source is diverted into systemic absorption.

According to another aspect, power consumption for the system is reduced. Power is required to switch the device from diversion mode to delivery mode, but little if any power is required to operate in diversion mode, which is the generally predominate mode of operation. If a power supply for the system, typically a battery, becomes depleted or other problem arises, the system operates in the diversion mode, which is a failsafe mode because drug is diverted away from the target sites, thereby enhancing the subject's safety. Because the system consumes less power, the battery will last longer or a smaller battery can be substituted.

An implantable flow regulator for regulating flow of drug along a drug delivery pathway from a source or drug delivery device to a treatment site within a subject includes a movable diversion member and an actuator. The movable diversion member is operably coupled to the delivery pathway. The actuator is actuatable to move the diversion member between at least a first mode position where the diversion member restricts flow through the delivery pathway (thus diverting flow through a diversion pathway), and a second mode position where the diversion member is positioned to allow flow through the delivery pathway. Supplying power to actuate the actuator moves the diversion member from its normally biased first mode position (diversion mode, delivery pathway “closed”) toward its second mode position (delivery mode, delivery pathway “open”).

The actuator may use any source of energy, e.g., electrical, mechanical, chemical and/or pneumatic energy, in functioning to move the movable member. In some of the described embodiments, the actuator is an electrical actuator (e.g., a solenoid), and it converts electrical energy received from an electrical power supply (e.g., a battery) into the mechanical energy necessary to move the movable member.

### BRIEF DESCRIPTION OF THE DRAWINGS

FIGS. 1A, 1B and 1C are schematics showing implantable drug delivery systems for delivering drug from a drug delivery device to a desired treatment site via a passageway with a flow regulator positioned to regulate the flow of the drug.

FIG. 2 is a sectioned side view of a first implementation showing an implantable flow regulator in a first mode in which flow from the source is being diverted to a waste vessel or systemic absorption.

FIG. 3 is a sectioned side view similar to FIG. 1, except showing the device in a second mode and a treatment site to which drug from the source is being directed.

FIG. 4 is a sectioned side view of a second implementation showing an implantable flow regulator that shares some of the characteristics of the device of FIGS. 1 and 2.

FIGS. 5A and 5B are sectioned side views of an implantable flow regulator according to a third implementation in first and second mode positions, respectively.

FIGS. 6A and 6B are sectioned side views of an implantable flow regulator according to a fourth implementation in first and second mode positions, respectively.

FIGS. 7A, 7B and 7C are schematic timing charts showing the interrelationship between the accumulation chamber volume, the delivery flow rate and the diversion flow rate.

### DETAILED DESCRIPTION OF THE PREFERRED EMBODIMENTS

Before the present methods and devices are described, it is to be understood that the particular implementations described and illustrated are not limiting, and as such may, of course, vary. It is also to be understood that the terminology used herein is for the purpose of describing particular implementations only, and is not intended to limit the scope of the present invention which will be limited only by the appended claims.

It must be noted that as used herein and in the appended claims, the singular forms “a,” “an,” and “the” include plural referents unless the context clearly dictates otherwise. Thus, for example, reference to “a formulation” includes mixtures of different formulations, and reference to “the method of delivery” includes references to equivalent steps and methods known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of the ordinary skill in the art to which this subject matter belongs. Although any methods and materials similar or equivalent to those described herein can be used in the practice or testing of the present invention, the preferred methods and materials are now described. All publications mentioned herein are incorporated herein by reference to disclose and describe the specific methods and/or materials in connection with which the publications are cited.

The publications discussed herein are provided solely for their disclosure prior to the filing date of the present application. Nothing herein is to be construed as an admission that the present application is not entitled to antedate such publication by virtue of prior invention. Further, the dates of publication provided may be different from the actual publication dates that may need to be independently confirmed.

### Description of Claim Terms

“Drug delivery system” is meant to refer to any device or combination of devices that can provide for transfer of drug from a drug reservoir to a treatment site. “Drug delivery device” thus encompasses, for example, a source or drug delivery device (e.g., an implantable pump); a flow regulator; the structures for delivery and diversion pathways (e.g., catheters, conduits, etc.); and the like.

The term “treatment site” as used herein is meant to refer to a desired site for delivery of drug from a drug delivery device of the invention. “Treatment site” is thus meant to include, although is not necessarily limited to, a subcutaneous, percutaneous, intravenous, intrathecal, intramuscular, intra-arterial, intravascular, intraperiotoneal, intraspinal, epidural, intracranial, peritumoral, or intratumoral (e.g., within a cancerous growths) site within a subject, as well as sites within or near a selected organ or tissue (e.g., central nervous system (e.g., intraspinal (e.g., epidural, intrathecal, etc.) within the spinal fluid, brain, etc.), peripheral nervous system, kidney, liver, pancreas, heart (e.g., intrapericardial), lung, eye, ear (e.g., inner ear), lymph nodes, breast, prostate, ovaries, testicles, thyroid, spleen, etc.), digestive system (e.g., stomach, gastrointestinal, etc.), skeletal muscle, bone, urinary bladder, gall bladder, adrenal gland, adipose tissue, parathyroid gland, uterus, fallopian tube, skin, into a vessel associated with the circulatory system (e.g., artery, arteriole, blood vessel, vein, capillary bed, lymph vessel, particularly arteries that feed a selected organ or tissue)), a tumorous growth (e.g., cancerous tumor (e.g., solid tumor), cyst, etc.), at a site associated with a microbial infection (e.g., bacterial, viral, parasitic or fungal infection), or to an autologous or synthetic graft (e.g., a vascular graft).

The term “subject” is meant any subject, generally a mammal (e.g., human, canine, feline, equine, bovine, etc.), to which drug delivery is desired.

The terms “drug,” “therapeutic agent,” or “active agent” as used herein are meant to encompass any substance suitable for delivery to a treatment site of a subject, which substances can include pharmaceutically active drugs, as well as biocompatible substances that do not exhibit a pharmaceutical activity in and of themselves, but that provide for a desired effect at a treatment site, e.g., to flush or irrigate a treatment site (e.g., saline), provide for expression or production of a desired gene production (e.g., pro-drug, polynucleotide, and the like), etc. In general, “drug” and the like are used to encompass any drug administered by parenteral administration, particularly by injection (e.g., intravascularly, intramuscularly, subcutaneously, intrathecally, etc.). Drugs compatible for delivery using the described devices and methods are discussed below, and are readily apparent to the ordinary skilled artisan upon reading the disclosure provided herein. Drugs may optionally be provided in combination with pharmaceutically acceptable carriers and/or other additional compositions such as antioxidants, stabilizing agents, permeation enhancers, etc.

The term “treatment” is used here to cover any treatment of any disease or condition in a mammal, particularly a human, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the disease and/or its symptoms.

### Overview

Described below are methods and devices for regulating the rate of drug delivery to a target site or sites. For convenience, only a single treatment site is shown in the figures, but the same concepts apply equally to a system in which flow of drug is directed to multiple treatment sites.

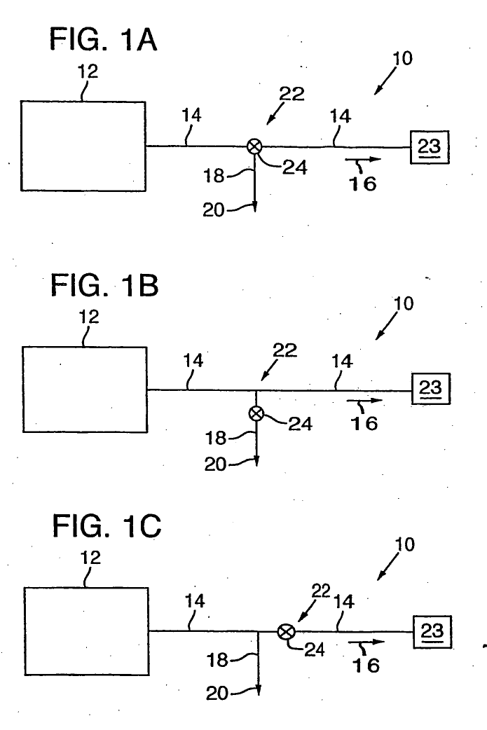

As illustrated schematically in FIGS. 1A-1C, a drug delivery system 10 includes a flow regulator 22 that is implanted within a subject to regulate drug delivery from one or more drug delivery devices to a treatment site. A first drug delivery device 12 and a first treatment site 23 are shown in the figures. With the system 10, flow of drug is controllably diverted away from a primary drug delivery pathway 14 (flow direction indicated by arrow 16) and into a diversion pathway 18 (flow direction indicated by arrow 20) with the flow regulator 22 according to, e.g., a predetermined treatment plan.

In general, the flow regulator 22 may include (1) a delivery conduit, which defines (at least in part) the delivery pathway 14 that flows toward a treatment site during use, and (2) a diversion element 24 (represented schematically by a valve symbol), which is a structural member operably coupled to the delivery conduit that facilitates diversion of drug flow from the delivery pathway 14, e.g., out of the delivery conduit, through a first outlet. In other embodiments, the flow regulator 22 includes a diversion conduit, which is selectively placed in fluid communication with the delivery conduit via a second outlet to define the diversion pathway 18 that diverts flow away from the portion of the delivery pathway 14 leading to the treatment site. For clarity, the majority of implementations described herein include both a delivery conduit and a diversion conduit, but the system is not meant to be so limited.

The flow regulator 22 can be provided in a variety of embodiments. For example, the diversion element 24 of the flow regulator can be positioned at the juncture of the delivery and diversion pathways (see, e.g., FIG. 1A), at a site of the delivery pathway distal to the diversion outlet (see, e.g., FIG. 1B), or, where the flow regulator comprises a diversion conduit that defines the diversion pathway, the diversion element can be positioned along the body of the diversion conduit (see, e.g., FIG. 1C).

In some embodiments, drug diverted into the diversion pathway 18 can be delivered to a site within the subject where the drug will have few or no undesirable side effects, e.g., to a site in the body away from the site of action of the drug. These embodiments are particularly useful where there is a local advantage to delivery of drug to a treatment site, which local advantage can be due to, for example, delivery of drug to directly to the desired site of action (e.g., to avoid side effects associated with systemic delivery), concentration effects (e.g., site-specific delivery provides for a drug concentration at the treatment site that is difficult or undesirable to accomplish through systemic delivery routes), and/or characteristics of the drug itself (e.g., short half-life, inactivation in the systemic absorption, etc.). These embodiments provide an elegant means for regulating drug delivery rate by taking advantage of the difference in the amount of drug that elicits a biological effect when delivered systemically. These embodiments take advantage of this difference in relative therapeutic thresholds to use the systemic absorption as a “waste reservoir” for drug diverted from a drug delivery pathway that targets a specific treatment site.

In other embodiments, the diverted drug is collected in a waste reservoir. These embodiments are particularly useful where the drug delivery system is for systemic drug delivery, i.e.; the rate of systemic drug delivery can be regulated by diverting the drug into a waste reservoir.

Specific exemplary embodiments of the invention are described below in more detail. The embodiments described below and in the figures are only exemplary and are not meant to be limiting in any way.

First Implementation

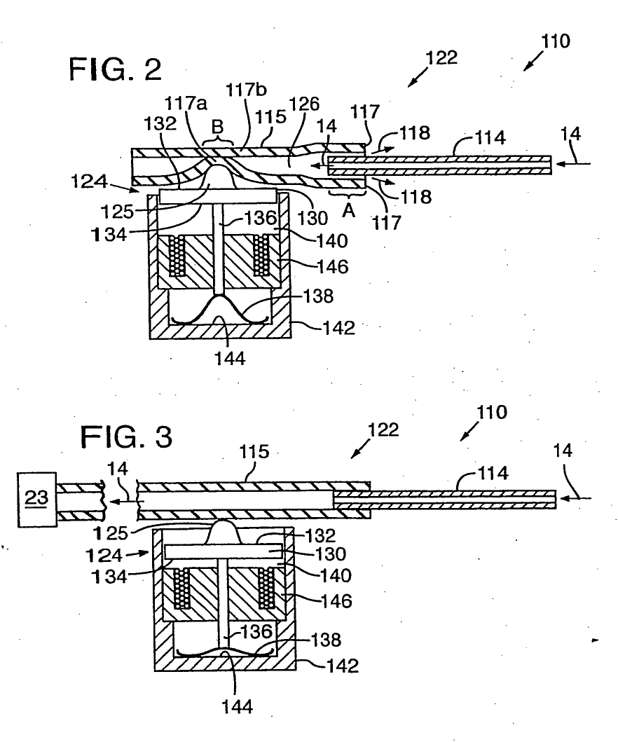

Referring to FIGS. 2 and 3, a system 110 according to a first implementation has a flow regulator 122 with a selectively actuatable diversion element 124 movable to change the flow through the primary drug delivery pathway 14 (indicated schematically by the arrows as shown). As illustrated, the delivery pathway 14 extends from a drug delivery device or source at the right (not shown), leftward through the flow regulator 122, and leftward to a treatment site (shown in FIG. 3). In this implementation, the drug delivery pathway 14 is defined by a delivery conduit first portion 114 and a downstream delivery conduit second portion 115 overlapped with the first portion 114 at a region A. The second portion 115 is typically formed of a resilient material (e.g., silicone), and is typically more flexible than the first portion 114.

FIG. 2 shows the flow regulator 122 in a first mode with the diversion element 124 positioned to block flow through the delivery pathway 14. Specifically, the diversion element 124 is in contact with the second portion 115 and deforming an adjacent side 117a into contact with an opposite side 117b, effectively “pinching off” the second portion 115 at an area B and preventing flow through it.

As shown in FIG. 2, the diversion element 124 may have a rounded projecting tip 125 to facilitate deforming the second portion 115 at a specific location (thus reducing the required force) while preventing damage to the second portion 115. Although not shown in FIGS. 2 and 3, it may desirable to position the opposite side 117b of the second portion 115 adjacent a stationary object to provide a surface against which the diversion element 124 can bear when moved into the position shown in FIG. 2.

An accumulation chamber 126 is formed upstream of the area B when the delivery pathway 14 is restricted. With the delivery pathway 14 restricted, additional flow entering the chamber 126 at a supply pressure P_{S} increases a pressure P_{C} within the chamber 126. When the pressure P_{C} reaches a predetermined threshold resistance pressure P_{R} (which is typically less than P_{S}), the pressure P_{C} is sufficient to deform an upstream end 117 of the second portion 115, causing it to expand and form a gap relative to the first portion 114, thereby establishing the diversion pathway 118. Accumulated fluid within the chamber leaks through the gap to the surroundings, thereby causing the pressure P_{C} to decrease. When the pressure P_{C} decreases below P_{R}, the upstream end 117 relaxes back into contact with the first portion 114 and seals the accumulation chamber 126. Additional fluid entering the accumulation chamber 126 from the first portion 114 causes the pressure P_{C} to rise again, and the above process is repeated.

In this implementation, the diversion pathway 118 leads away from the specific treatment site or sites. For example, the diversion pathway 118 may divert excess accumulated drug into systemic absorption or to a part of the body where the drug has no effect.

FIG. 3 shows the flow regulator 122 in a second mode in which the diversion element 124 is positioned to allow flow through the delivery pathway 14 and to the treatment site 23. Specifically, the diversion element 124 has been retracted from the first mode position shown in FIG. 2 to allow the pinched second portion 115 to at least partially open, thereby establishing the delivery pathway 14 to the treatment site 23.

Operation

In the illustrated implementations, the flow regulator 122 is normally in the first mode position, i.e., with the diversion element 124 restricting the delivery pathway 14 and flow from the second portion following a path into the accumulation chamber 126 and out the diversion pathway 18. At predetermined intervals or according to another treatment plan, the flow regulator 122 is actuated, the delivery pathway 14 to the treatment site 23 opens, and a desired small amount of drug, called a bolus, flows through the delivery pathway 14 to the treatment site 23.

The delivery pathway 14 to the treatment site 23 includes the accumulation chamber 126. When the second portion 115 has been released as shown in FIG. 3, drug flows from the accumulation chamber 126 leftward toward the target site 14. Following the delivery interval and after the second section is pinched closed (see FIG. 2), the constant flow of drug to the regulator 122 begins to reestablish the reserve within the accumulation chamber 126. After the predetermined reserve volume accumulates, additional flow is diverted along the diversion pathway 118.

Delivery can be controlled by varying the duty cycle of the associated circuitry that switches the actuator on and off. For example, a constant-rate osmotic pump (not shown) may be connected as the drug source or delivery device. Such a pump, which may be activated by salt within the subject, supplies drug to the system at a substantially constant rate. Under many treatment plans, only small amounts of drug are delivered, so the remaining drug supplied to the system is diverted.

Construction Details

In the implementation of FIGS. 2 and 3, the diversion element 124 is a piston-like member 128 having a disk portion 130, with a first side 132 from which the tip 126 protrudes and a second side 134 from which a shaft 136 extends. As shown, the diversion element 124 is positioned within a recess 140 defined by a housing 142.

A spring 138 is positioned at the distal end of the shaft 136. The housing 142 has a base 144 against which the spring 13S bears. As shown in FIG. 2, the diversion element 124 is biased by the spring 138 toward the first mode position.

Within the housing 142, there is an actuator element 146, in this case an electrical actuator element (e.g., a solenoid), that is selectively actuatable to retract the diversion element 124 from its normal spring-biased first mode position to its second mode position. As illustrated, such an actuator element 146 typically has a coil connected to a power source (not shown) that can be selectively energized to create a magnetic field of sufficient strength to overcome the force of the spring 138 and retract the diversion element 124 toward the base 144. Other types of actuator elements, e.g., those that use electrical, mechanical, chemical and/or pneumatic energy, can also be used.

In other implementations where more continuous flow regulation is desired, it is possible to control the diversion element 124 to deform the second portion 115 to reduce the internal flow area and thereby restrict flow without terminating it completely.

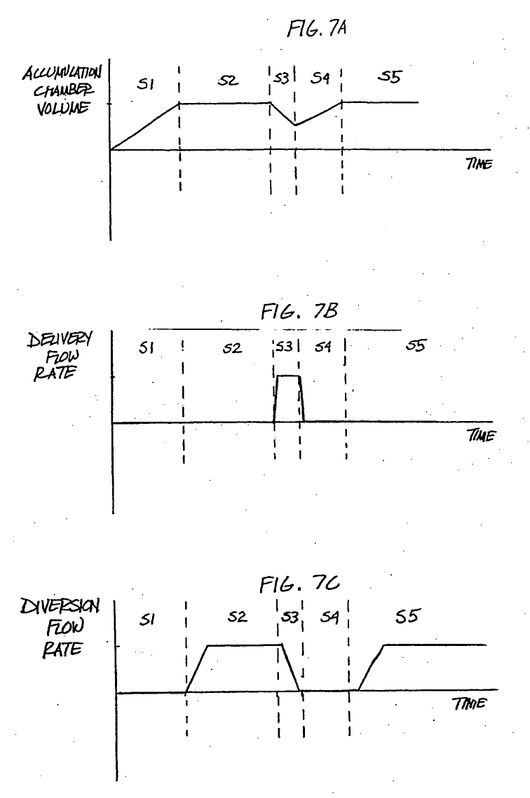

Timing charts showing the interrelationship during operation between the accumulation chamber volume, the delivery flow rate and the diversion flow rate are illustrated in FIGS. 7A, 7B and 7C.

Referring to FIG. 7A, a segment S1 shows the accumulation chamber volume increasing from zero to the predetermined reserve volume (i.e., “full”), which occurs upon initial filling after initialization and possibly if the system is re-initialized In the segment S2, the accumulation chamber remains substantially constant at the full reserve volume. This represents, e.g., steady state operation in which drug received at the regulator is being diverted.

Segment S3 shows the accumulation chamber volume decreasing, e.g., after transition from diversion mode to delivery mode. The volume decreases from the full reserve volume level, but does not entirely deplete the reserve. Segment S4 shows the accumulation chamber volume increasing, e.g., after the supply rate to the regulator begins to exceed the delivery rate, such as after transition from delivery mode back to diversion mode. The volume increases until the full reserve volume level is reached, as shown in segment S5.

Referring to FIG. 7B, segments S1 and S2 show a zero rate of delivery of drug from the regulator to the target site, e.g., while the system is operating in diversion mode. In segment S3, e.g., after transition from diversion mode to delivery mode, the delivery rate rises from zero sharply to a maximum delivery rate, which remains substantially constant, and then decreases back to zero. The delivery rate remains at zero, as shown in segments S4 and S5, until the next delivery mode interval.

Referring to FIG. 7C, the diversion flow rate is zero during segment S1, throughout initialization, until the accumulation chamber reaches the full volume level. After the accumulation chamber reaches the full volume level, normal diversion mode commences, with drug supplied to the regulator being diverted from the regulator. Following initiation of delivery mode, the diversion rate decreases. For the purposes of illustration, the diversion rate is shown decreasing to zero in segments S3 and S4. Following completion of a delivery mode interval, the diversion flow increases to maximum rate as shown in segment S5.

Second Implementation

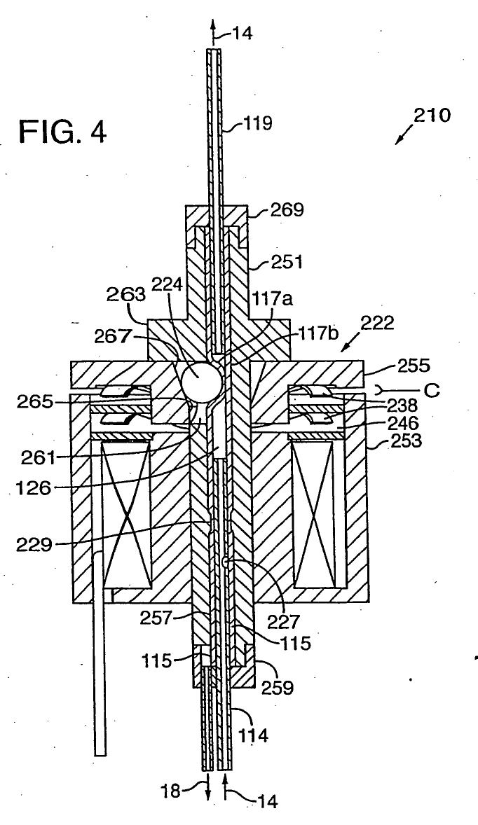

According to a second implementation as shown in FIG. 4, a system 210 is configured with the delivery pathway 14 extending through a body of a flow regulator 222 and defining an axial direction.

FIG. 4 shows the flow regulator 222 in the first mode position with the delivery pathway 14 blocked by a diversion element 224 that has “pinched off” the second portion 115. As illustrated, the diversion element 224 can be a sphereoid (commonly referred to as a “ball”) or other suitable shape that is moved radially relative to the delivery pathway 14, eventually reaching the position shown in FIG. 4 and deforming the adjacent side 117a of the second portion 115 into contact with the opposite side 117b.

In this implementation, a movable element 255 moves in a direction generally parallel to the flow direction through the flow regulator 222. Stated differently, the actuator element 255 moves in a direction generally perpendicular to the direction in which the diversion element 224 moves.

The flow regulator 222 has a generally cylindrical body with a fixed inner member 251 and a fixed outer member 253. The movable outer member 255 is coupled to the body.

The inner member 251 has a bore 257 sized to accommodate the second portion 115. The first portion 114 extends through an end 259 of the body and into the second portion 115 as shown. The inner member 251 also has a transverse opening 261 in a side of the bore 257 and within which the diversion element 224 is free to move.

The fixed outer member 253 is fitted to an outer surface of the inner member 251. The fixed outer member, which is axially opposite the movable member 255 as shown, houses the actuator 246.

In the illustrated implementation, the movable outer member 255 is coupled to an outer surface of the inner member 251 by sliding engagement. The movable outer member 255 moves axially relative to the inner and fixed outer members 251, 253. The movable outer member 255 is biased away from the fixed outer member, e.g., by one or more springs, such as a pair of helical springs 238. In the first mode position, the movable outer member bears against a collar 263 formed on the inner member 251 that maintains the movable outer member 255 at a fixed distance C from the stationary outer member 253.

In the illustrated implementation, the movable outer member is ring-shaped with an axial opening 265 having a sloping sidewall 267 that flares outwardly to give the opening 265 a generally frustoconical shape in section, with the base of the cone facing the collar 263. As shown in FIG. 4, when the actuator 246 is actuated to draw the movable outer member 255 toward the fixed outer member 253, the diversion element 224 is permitted to move axially outward while remaining in contact with the sidewall 267. When the movable outer member 255 is in the second mode position, pressure in the accumulation chamber 126 is sufficient to force the sides of the second portion 115 apart so that the delivery pathway to a site or sites is established. As shown in FIG. 4, there is a delivery outlet tube extending through an end 269 of the body and toward the treatment site.

When the flow regulator 222 is in the first position as shown in FIG. 4, continued flow of drug through the first portion 115 will cause the pressure within the accumulation chamber 126 to increase. The accumulation chamber may expand slightly beyond the position shown in FIG. 4, e.g., into the area of the opening 261. When a threshold pressure is reached, flow from the first portion at an outlet opening 227 is forced between the second portion 115 and the first portion 114, and travels in a direction generally opposite the delivery pathway 114. An internal rib 229 in the bore 257 crimps the second portion 115 against the first portion 114 and restricts flow from the outlet opening 227 from continuing in this direction.

The flow from the outlet opening 227 travels toward the end 259 and exits from between the first portion 114 and the second portion 115 through an opening 271 formed at a junction between the inner member 251 and the end 259. A radially offset outlet tube diversion tube 273 in communication with the opening 271 directs flow from the opening out of the flow regulator 224 and to, e.g., systemic absorption or a waste vessel.

According to one configuration, the axial distance C is less than about 1 mm and the actuator is actuated for about 1 second, and possibly as long as two seconds, at a time, according to a typical treatment plan.

Third Implementation

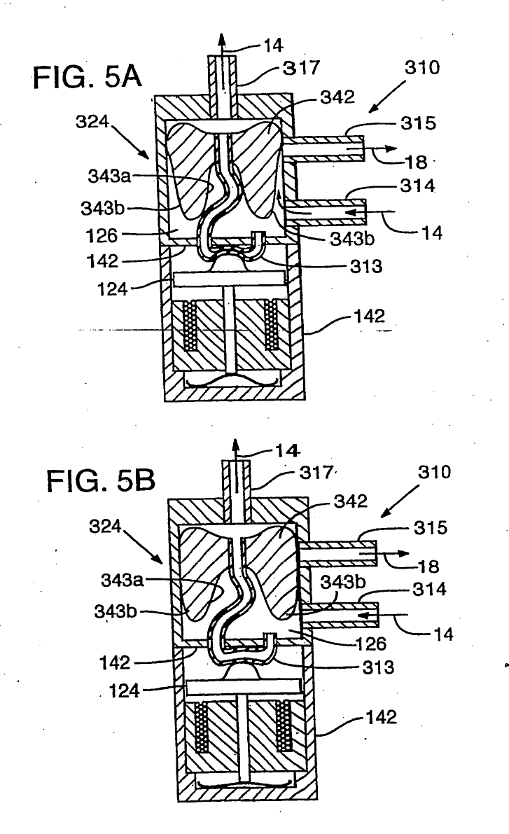

FIGS. 5A and 5B show a system 310 according to a third implementation. A flow regulator 324 includes a resilient member or bellows 342 positioned at one end of the accumulation chamber 126 and shaped to deform under pressure exerted by fluid in the accumulation chamber 126.

As shown in FIG. 5A, the flow regulator 324 is in the first mode position in which the drug delivery pathway 14 is interrupted and no drug is flowing to the treatment site. The drug delivery pathway to the flow regulator 322 is defined by an inlet tube 314 leading to and in communication with the accumulation chamber 126. Entering fluid increases the pressure within the accumulation chamber 126. The member 342 is shaped such that a threshold pressure on an inner portion 343a causes an outer portion 343b to converge slightly inwardly and separate from the adjacent wall as shown, thereby allowing flow through the diversion pathway 18 through a diversion outlet 315.

FIG. 5B shows the system 310 in the second mode when the diversion element 124 has been retracted. A flow is established from the accumulation chamber 126 and through a flexible tube 313 to establish the drug delivery pathway 14 to the treatment site or sites. The flexible tube 313, which in the first mode is pinched closed by the diversion element 124 (FIG. 5A), extends from a first end that communicates with the accumulation chamber 126, through a partition 143 in the housing 142 that defines a boundary of the accumulation chamber 126, and through the member 342. The flow then exits through an outlet tube 317 extending from an end of the housing 142.

The opening of the tube 313 provides an easier path for flow, and flow through the tube 313 decreases the pressure applied to the member 342. The decrease in pressure allows the member 342 to relax against the adjacent wall and effectively seal off the diversion outlet 315.

Advantageously, the member 342 can deform under pressure to seal off flow from exiting through the diversion conduit 315 without requiring any separate seal or gasket. As a result, the member 342 and the adjacent interior walls together form a gasket-free seal. Since a separate gasket is not required, the system is easier to maintain and requires less maintenance.

Fourth Implementation

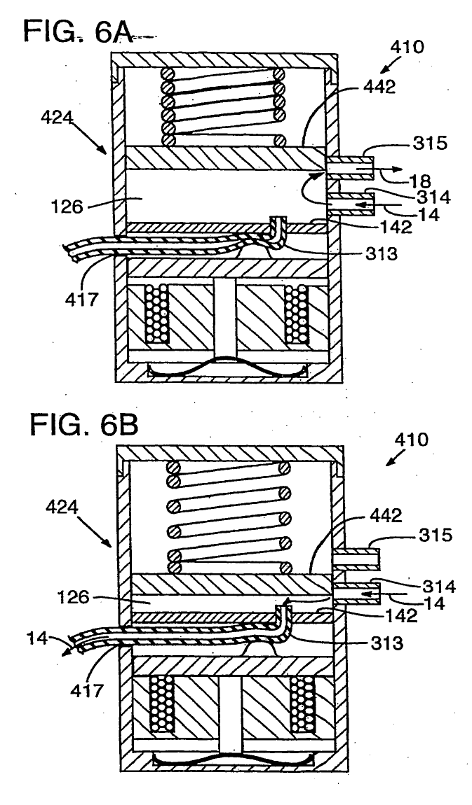

FIGS. 6A and 6B show a system 410 according to a fourth implementation. The system 410 includes a flow regulator 424. The flow regulator 424 is similar to the flow regulator 324 described above, except the flow regulator 424 includes a second spring-biased piston 442 instead of the resilient member 342. Similar to the flow regulator 324, the flow regulator 424 allows the volume of the accumulation chamber 126 to expand under pressure within a predetermined volume range.

In the first mode position as shown in FIG. 6A, the volume of reserve drug within the accumulation chamber has reached a predetermined volume limit. Pressure within the accumulation chamber 126 has moved the piston 442 sufficiently away from the partition 142 to expose the diversion outlet 315 and allow accumulated drug to flow out of the accumulation chamber 126. Thereafter, the piston 442 retracts toward the partition 142 under the action of the spring (see FIG. 6B), and the process is repeated.

In the second mode when the diversion element 124 is retracted as shown in FIG. 6B, a flow is established from the accumulation chamber 126 to establish the drug delivery pathway 14 to the treatment site or sites. In the system 410, the flexible tube 313 leads from the accumulation chamber 126 through the partition 142 and out of the flow regulator 424, in this case through an opening 417 in a side wall of the regulator 424. A seal (not shown) may be provided around the piston 442 to seal against substantial leakage.

In general, the system takes advantage of directing substantially all of the drug received at the flow regulator to either the target site (i.e., in delivery mode) or to systemic absorption (i.e., in diversion mode), which can be used to assist in determining how much drug is delivered or not delivered. Under this approach, measuring any one quantity allows the other to be determined with acceptable precision. It would also be possible for the regulator to have a residual diversion flow while in delivery mode, i.e., a small amount of flow from the regulator into systemic absorption while a substantial majority of flow from the regulator is being directed to the target site.

In general, the various components of the flow regulator may be made of any bio-compatible material. Stainless steels, titanium alloys and plastics are possible materials. If the actuator is a solenoid that generates a magnetic field, the disk portion 130, the movable member 255 and corresponding components of other embodiments must be made of a ferromagnetic material, e.g., a ferromagnetic stainless steel. The resilient portion 115 of the delivery conduit may be formed of a flexible silicone as stated, or urethane or other similar material.

Having illustrated and described the principles of our invention with reference to several implementations, it should be apparent to those of ordinary skill in the art that the invention may be modified in arrangement and detail without departing from such principles.

We claim:

1. A flow regulator implantable within a subject for regulating flow of a drug through a delivery pathway from a source to a treatment site, the flow regulator comprising:
a movable diversion member operably coupled to the delivery pathway;
an actuator actuatable to move the diversion member between at least a first mode position and a second mode position;
the first mode position being defined where the diversion member is substantially restricting flow through the delivery pathway;
the second mode position being defined where the diversion member is positioned to allow flow through the delivery pathway,
wherein the diversion member is biased to normally return to the first mode position and restrict flow to the treatment site, and wherein supplying power to actuate the actuator moves the diversion member from the first mode position toward the second mode position.

2. The flow regulator of claim 1, wherein an accumulation chamber is defined upstream of the diversion member, and wherein when the diversion member is in the first mode position, a reserve of drug can be accumulated in the accumulation chamber from drug flowing from the source to the regulator.

3. The flow regulator of claim 1, wherein moving the diversion member from a first mode position to a second mode position causes flow through the delivery pathway from a reserve of drug accumulated within the flow regulator.

4. The flow regulator of claim 1, wherein the delivery pathway includes a delivery conduit with a resilient portion and the diversion member is movable to deform the resilient portion such that flow through the resilient portion is restricted.

5. The flow regulator of claim 1, wherein the diversion member is movable in a direction substantially perpendicular to a direction of flow through the delivery pathway.

6. The flow regulator of claim 4, wherein the diversion member has a first end movable into contact with the resilient portion and an opposite second end, the flow regulator further comprising a spring positioned adjacent the second end to bias the diversion member toward the first mode position.

7. The flow regulator of claim 4, wherein a portion of the diversion member that contacts the resilient portion is rounded, and the diversion member in the first mode position pinches off the resilient portion.

8. The flow regulator of claim 1, wherein the delivery pathway includes at least a delivery conduit first portion and a delivery conduit second portion, the second portion being made of a resilient material and having an upstream end positioned to overlap a downstream end of the first portion and wherein the upstream end of the second portion is deformable from pressure within the second portion such that the upstream end can separate from the downstream end of the first portion and allows fluid from within the second portion to escape.

9. The flow regulator of claim 1, wherein the diversion element has a spheroidal shape.

10. A flow regulator implantable within a subject for regulating flow of a drug through a delivery pathway from a source to a treatment site within the subject, the flow regulator comprising:
a generally cylindrical body in which a generally axial bore is defined;
the bore having a generally transverse opening defined therein;
a delivery conduit that forms a part of the delivery pathway and extends through the bore, the delivery conduit having a resilient portion positioned adjacent the opening in the bore;
a spheroidal diversion element positioned within the opening and adjacent the resilient portion;
a movable member coupled to the body adjacent the diversion element and the opening in the bore and moveable in the axial direction to change the position of the diversion element among at least first and second positions;
the first position being defined where the diversion element is urged radially inwardly against the resilient portion and delivery pathway is restricted;
the second position being defined at a point axially spaced from the first position where the diversion element is free to move radially away from the resilient portion such that flow can pass through the delivery conduit; and
an actuator positioned within the body and actuatable to move the movable member from the first position in the axial direction.

11. The flow regulator of claim 10, wherein the resilient portion of the delivery conduit is a second portion, and the delivery conduit includes a first portion inserted into an upstream end of the second portion, the delivery conduit having an overlapped region in which the second portion overlaps the first portion in the axial direction.

12. The flow regulator of claim 11, wherein the first portion has a side wall in which a transverse outlet opening is defined within the overlapped region.

13. The flow regulator of claim 12, wherein an accumulation chamber is defined by a space within the delivery conduit from the outlet opening in the first portion downstream to an area where the diversion member contacts the delivery conduit when the movable member is in the first position.

14. The flow regulator of claim 13, wherein a diversion pathway is defined from within the first portion, through the outlet opening in the first portion and between the first portion and the second portion.

15. The flow regulator of claim 14, further comprising a radially offset outlet tube extending from the body, and wherein the diversion pathway between the first portion and the second portion continues in a downstream direction through the outlet tube.

16. The flow regulator of claim 10, wherein the movable member has a generally axial opening dimensioned to receive the body.

17. The flow regulator of claim 10, wherein the movable member is ring-shaped with a generally axial opening dimensioned to slidingly engage an outer side of the body.

18. The flow regulator of claim 10, wherein the movable member is spring-biased toward the first position.

19. The flow regulator of claim 1S, further comprising at least one helical spring positioned to urge the movable member toward the first position.

20. The flow regulator of claim 10, wherein the body includes a stop portion adjacent the transverse opening, and the movable member contacts the stop member when the movable member is in the first position.

21. The flow regulator of claim 10, wherein the body includes a fixed inner part and a fixed outer part that surrounds the inner part, and wherein the fixed outer part houses the actuator and is positioned axially opposite the movable member.

22. The flow regulator of claim 10, wherein the actuator is a solenoid that generates a magnetic field when actuated, and wherein the movable ring is made of a ferromagnetic material.

23. The flow regulator of claim 10, wherein the resilient portion is made of a silicone material.

24. The flow regulator of claim 10, wherein the delivery pathway includes a delivery outlet tube connected to a downstream end of the resilient portion and extending through an end of the body.

25. The flow regulator of claim 10, wherein an axial distance between the first and second positions is less than about 1 mm.

26. An implantable drug delivery system for delivering drug to a treatment site within a subject according to a predetermined treatment plan, comprising:
an implantable drug source capable of supplying drug at a substantially constant rate;
an implantable flow regulator that is connectable to the source to receive drug from the source;
an accumulation chamber defined in the flow regulator and sized to hold a reserve of drug received from the source;
a first outlet on the flow regulator defining part of a diversion pathway along which drug can be diverted to a waste vessel or into systemic absorption;
a second outlet on the flow regulator defining part of the delivery pathway to the treatment sites;
the flow regulator having a first mode of operation in which the accumulation chamber is filled and thereafter drug is diverted through the first outlet and along the diversion pathway, and a second mode in which drug flows from the accumulation chamber outward through the second outlet and along the delivery pathway.

27. The implantable drug delivery system of claim 26, wherein in the second mode, drug flow from the accumulation chamber flows at a substantially constant rate through the second outlet.

28. A method of supplying drug on demand in an implantable drug delivery system, comprising:
providing a drug delivery source implanted within a subject containing drug to be delivered to a treatment site;
providing a flow regulator implanted within a subject in fluid communication with and positioned downstream of the source, the regulator having an accumulation space defined therein in which a reserve of drug from the source is accumulated, the regulator having a first mode position in which drug from the source is diverted away from the treatment site and a second mode position in which drug is delivered to the treatment site; and
supplying drug to the treatment site on demand from the accumulation space after the regulator is changed from the first mode position to the second mode position.

29. The method of claim 28, further comprising regulating the volume of drug in accumulation space by diverting drug along a diversion path when the accumulation space is filled to a predetermined capacity.
